(19) United States
(12) Patent Application Publication
(10) Pub. No.: US 2023/0310497 A1
(43) Pub. Date: Oct. 5, 2023

(54) DISINFECTING, SKIN-TOLERABLE CARE AGENT FOR APPLICATION TO THE SKIN

(71) Applicants: TULICHEM GMBH, Mainz (DE); MERLIN APOTHEKE RUTH GEBHARDT UND DR. FRANK ULLROCH OHG, Sankt-Augustin (DE)

(72) Inventors: Rainer Pommersheim, Mainz (DE); Denis Breuch, Mainz (DE)

(21) Appl. No.: 18/011,607

(22) PCT Filed: Jun. 24, 2021
(86) PCT No.: PCT/EP2021/067357

§ 371 Date: Dec. 20, 2022

(30) Foreign Application Priority Data
Jun. 30, 2020 (DE) 20 2020 103 775.7
Jul. 24, 2020 (DE) 20 2020 104 277.7

(51) Int. Cl. A61K 33/20; A61K 31/145; A61K 8/29; A61Q 17/04

(52) U.S. Cl. CPC A61K 33/20; A61K 31/145; A61K 8/29; A61Q 17/04

(57) ABSTRACT

The invention relates to a disinfecting, skin-tolerable care agent for application to the skin or for the treatment thereof, comprising one or more water-soluble chlorine derivatives as a disinfecting active substance, wherein the chlorine derivative(s) is/are incorporated in a base creme or gel matrix and additionally contain(s) a substance mixture for odor-masking the chlorine component.

The invention relates to a disinfecting, skin-tolerable care agent for application to the skin and for the treatment thereof, comprising one or more water-soluble chlorine derivatives as a disinfecting active substance, a creme on the basis of such an agent, as well as a dispenser, a tin, a tube or a package with such a disinfecting agent of a respective creme or a gel according to the teaching of the independent claims.

Products are known which can be applied to the skin and which result in a disinfecting, germ-reducing effect due to the application to the skin. Known preparations are based on the active substances isopropyl, ethanol or other alcohols.

Apart from liquid products, gels are known, in which the alcohol has been thickened.

So as to develop a sufficient disinfecting effect here, the alcohol must be present in a concentration of at least 70%. Such a concentration can simply be realized in gels. A creme, however, cannot be fabricated with respect to these concentrations.

Hitherto known cremes containing alcohol for disinfection, therefore are not sufficient in the medical sense as well as in terms of hygienic aspects and limited in their effectiveness.

From the document DE 10 2006 043 245 A1, a chloramine-containing wound treatment agent is already known.

A 10% chloramine-containing wound compress was obtained there by spraying with a chloramine T solution. Together with a physically loaded zeolite component and a liquid permeable cover material, the introduced system can be able primarily for bleeding wounds at simultaneous disinfection by aqueous chloramine solution for treatment.

Reference is made in the document EP 1 955 592 A1 to a benzalkonium chloride as a disinfecting agent and antimicrobial skin treatment agent in a creme base with respect to the agent for care and for antimicrobially treating the skin. This creme has a single pharmaceutically active component based on the mentioned benzalkonium chloride. In an ointment in this respect, viscous to solid paraffine, wool wax and/or white vaseline is utilized. The agent according to EP 1 955 591 A1 is deemed to be also suitable for the caring disinfection of the skin, in particular for doctors or medical personnel.

From the aforementioned, it is a task of the invention to propose a further developed, disinfecting, skin-tolerable care agent, in particular a creme for being applied onto the skin, which displays, apart from the good skin-tolerability and caring and greasing effect, a verifiable sufficient, disinfecting effect, wherein a wound disinfection is primarily not concerned but the everyday use for hand disinfection and thus for interrupting potential infection chains.

From a medical point of view, a creme is understood to be an oil-in-water emulsion or a water-in-oil emulsion making ingredients ready for being applied. Relative to ointments, cremes are less viscous, i.e. are more liquid, and have a good absorption into the skin. In cremes, emulsifiers or emulsion-stabilizers are frequently used. In common parlance, cremes are also referred to as non-medical, purely caring products.

According to the depicted definition of task, it is solved by a disinfecting, skin-tolerable care agent for application to the skin or for treatment of the skin in accordance with the feature combination according to claim 1, a creme or a gel according claim 4, or a dispenser, a tin, a tube or a package according to claim 8.

In contrast to known solutions, the agent according to the invention does not count on an alcohol as a disinfection active substance, but on a chlorine derivative. Such an active substance is recommended and suitable in corresponding concentrations as a disinfecting agent when being applied to the skin.

The chlorine derivatives used in the formulation according to the invention are of good water-solubility and can be very well incorporated into the creme in the recommended concentrations.

Conducted tests with test persons which had been performed with the creme according to the invention prove the germicidal effect at simultaneous good skin-tolerance.

The disinfecting, skin-tolerable care agent accordingly is based on one or more chlorine derivatives as the disinfecting active agent.

The chlorine derivative(s) is/are incorporated in a base creme or a gel matrix.

In addition, the creme or the gel contains a substance mixture for odour-masking the chlorine component which otherwise can be perceivable.

In a developing manner, the agent has at least one fragrance or fragrance mixture.

In further developing the invention, a defined amount of functional active agents may be added to the prescribed formulation. This may be, for example, an active agent for humidifying or for protecting against harmful radiation, in particular ultraviolet radiation.

The creme according to the invention on the basis of the described agent is characterized in that the amount of the chlorine derivative used, in particular chloramine T, amounts essentially up to 5 weight-%, preferably and in particular up to 2 weight-%.

The base creme contains at least glycerides, vaseline and stearates. The gel matrix contains at least sodium polyacrylate or water.

The disinfecting component, i.e. the chlorine derivatives are, as already mentioned, chloramine T, sodium hypochlorite or similar substances.

The proposed agent or the corresponding creme is free from alcohols with respect to the disinfecting active agent. Degreasing of the skin as it is the case in usual liquid agents for disinfection does not take place.

According to the invention is a dispenser, a tin, a tube or a similar package with the proposed agent or a corresponding creme for different uses, in particular for application to the human skin.

The invention will be explained below in more detail by means of an exemplary embodiment.

The used base creme-matrix combines all of the ingredients required for skin care and secures the greasing and simultaneously caring effect of the creme.

The disinfecting active agent is based on water-soluble chlorine derivatives which are accepted disinfecting agents and fulfil the official regulations.

The substance mixture for odour-masking is a mixture of dermatologically approved components having the target of masking the chlorine odour released from the disinfecting active agent.

Usable odour substances confer a pleasant odour to the creme. These may be essential oils or other aromatic substances or mixtures produced therefrom.

In the functional active agents, for example, urea may be used so as to achieve an additional humidifying effect. Likewise, an agent for absorbing ultraviolet radiation may also be used.

A customary product may be used in particular for the base creme.

Such a base creme comprised the following exemplary ingredients:

Glycerol monostearate 60, cetyl alcohol, medium chain triglycerides, white Vaseline, macrogol-20-glycerol monostearate, propylene glycol and purified water.

For the disinfecting active agent, chlorine derivatives are utilized, such as, for example, chloramine T or sodium hypochlorite. In particular for chloramine T, the concentration in the creme amounts to 2 weight-%. This is a harmless upper limit giving a demonstrable sufficient disinfecting effect.

For the substance mixture for odour-masking, commercially available products may be referred to.

The following compositions are exemplary:

Ingredients Mixture A:

2,6-Dimethyloct-7-en-2ol, 3,7-Dimethyloctan-3-ol, 1-(1,2,3,4,5,6,7,8Octahydro-2,3,8,8tetramethyl-2naphthyl)ethan-1-one, Benzenepentanol, bmethyl-, Isopentyl salicylate, 3-(p-Ethylphenyl)-2,2dimethylpropionaldehyde, 3,7-Dimethyloctan-3-yl acetate, Benzenepropanol, .beta.,.beta.,3-trimethyl-, 2-Phenylethanol, Anisaldehyde, (E)-4-(2,6,6-Trimethyl1-cyclohexen-1-yl)-3buten-2-one, Oxacyclohexadecen-2-one, Piperonal, 1,4-Dioxacycloheptadecane5,17-dione, Pentadecan-15-olide, Hexyl acetate, 3-(o-Ethylphenyl)-2,2dimethylpropionaldehyde, 1-Methyl-4-(4methylpentyl)cyclohex3-ene-1-carbaldehyde 1;

Ingredients Mixture B:

Hexyl salicylate, 3,7-Dimethyloctan-3-ol, 1-(1,2,3,4,5,6,7,8Octahydro-2,3,8,8tetramethyl-2naphthyl)ethan-1-one, 2H-Pyran-4-ol, tetrahydro-4-methyl-2(2-methylpropyl)-, Benzenepentanol, bmethyl-, Benzenepropanol, .beta.,.beta.,3-trimethyl-, Pentadecan-15-olide, 2,6-Dimethyloct-7-en-2ol, α-Methyl-1,3benzodioxole-5propionaldehyde, 4-(4-Methyl-3pentenyl)cyclohex-3ene-1-carbaldehyde, (E)-Anethole, 2-Ethyl-4-(2,2,3trimethyl-3-cyclopenten1-yl)-2-buten-1-ol, 1,2,3,5,6,7-Hexahydro-1,1,2,3,3-pentamethyl-4H-inden-4-one, 3-(p-Ethylphenyl)-2,2dimethylpropionaldehyde, MUGETANOL, Piperonal, Oxacyclohexadecen-2-one, 4-Methyl-3-decen-5-ol, 3-(o-Ethylphenyl)-2,2dimethylpropionaldehyde, Allyl cyclohexyloxy)acetate, Methyl 2,4-dihydroxy-3,6-dimethylbenzoate, 2,6-Dimethylhept-5-enal, Octahydro-7-methyl-1,4methanonaphthalen6(2H)-one, cis-Hex-3-en-1-yl methyl carbonate

Essential oils, such as rose oil, lavender oil or similar, or mixtures of such essential oils are used as fragrances.

The mentioned functional active agents do not need to be components of the creme but can improve its effect and care for further applications.

As a means for protecting against ultraviolet radiation, a titanium dioxide can be added to the creme so as to ensure a corresponding light protection factor.

A creme ready for sale as a disinfecting, skin-tolerable and care agent comprises at least the base creme and the disinfecting active agent in the form of water-soluble chlorine derivatives.

By adding the substance mixture for odour-masking, the application is significantly better accepted since the unpleasant chlorine odour or the used chlorine derivate(s) is (are) subjected to encapsulation.

The creme according to the invention is filled after having been prepared and manufactured. Filling can be performed into a suitable dispenser, into a tin, a tube or a similar package.

The use of the creme takes place by application onto the skin and slight rubbing in, wherein the disinfecting and simultaneous care effect is deployed.

The introduced creme may be used either alone or for the subsequent care of the skin when disinfecting products on the basis of alcohol have been used before.

In the embodiment as a gel according to the invention, this is transparent or translucent in contrast to the introduced creme.

Both the creme and the gel according to the invention can be added further substances in an encapsulated form.

The beads configured in an encapsulated form generate a particular optical effect. In particular, the beads are of a different colour than the base creme or are optically striking relative to the transparent creme. The beads containing additional substances float within the gel or are distributed in the volume of the creme. In one example, the beads having the therein received encapsulated additives have a diameter of essentially 2 mm.

1. A disinfecting, skin-tolerable care agent for application to the skin or for the treatment thereof, comprising one or more water-soluble chlorin derivatives as a disinfecting active substance,
characterized in that
the chlorine derivative(s) is/are incorporated in a base creme or gel matrix and additionally contain(s) a substance mixture for odour-masking the chlorine component.

2. The disinfecting, skin-tolerable care agent according to claim 1,
characterized in that
it contains at least one fragrance or a fragrance mixture.

3. The disinfecting, skin-tolerable care agent according to claim 1,
characterized in that
it has functional active agents in a prescribed formulation, for example, for humidifying or for protecting against harmful radiation.

4. A creme or a gel on the basis of an agent according to claim 1,
characterized in that
the amount of the chlorine derivatives amounts essentially up to 5 weight-%, in particular up to 2 weight-%.

5. The creme on the basis of an agent according to claim 1,
characterized in that
the base creme contains at least glycerides, vaseline and stearates, and the gel matrix contains at least sodium polyacrylate and water.

6. The agent or creme according to claim 1,
characterized in that
chloramine-T, sodium hypochlorite or a similar chlorine derivative is contained as the disinfecting component.

7. The agent or creme according to claim 1,
characterized in that
it is free from alcohols with respect to the disinfecting active agent.

8. A dispenser, tin, tube or package having an agent or a creme according to claim 1.
